(19) United States
(12) Patent Application Publication
(10) Pub. No.: US 2007/0116742 A1
(43) Pub. Date: May 24, 2007

(54) Confectionary products containing a human pheromone component

(76) Inventors: Philip Y. Braginsky, Forest Hills, NY (US); Barry J. Marenberg, New Providence, NJ (US); Katey Lang, New York, NY (US)

Correspondence Address: Barry J. Marenberg, Esq., 76 Brookside Drive, New Providence, NJ, 07974, US

(21) Appl. No.: 11/342,174

(22) Filed: Jan. 28, 2006

Related U.S. Application Data
(60) Provisional application No. 60/738,890, filed on Nov. 22, 2005.

(51) Int. Cl. A61K 9/68; A61K 31/56; A61K 31/57

(52) U.S. Cl. 424/440; 514/178; 514/170

(57) ABSTRACT

Confectionary products, such as hard or soft candies, having a human pheromone compound as an ingredient of the product, and which causes a person eating the product to experience unique, mood altering effects. The effect may be an overall sense of well-being or one of heightened sexual attractiveness.

### RELATED APPLICATIONS

This application claims the benefit of U.S. Provisional Application No. 60/738,890, filed Nov. 22, 2005.

### FIELD OF THE INVENTION

The present invention relates to confectionary products and food items having a human pheromone component.

### BACKGROUND

Pheromones are chemicals emitted by living organisms to send messages to individuals of the same species. The classes most widely explored are the sex pheromones produced by female moths which are used to attract conspecific males for mating. Bombykol, the sex pheromone of the silkmoth, was first synthesized in 1959.

Most pheromones consist of blends of two or more chemicals which need to be emitted at exactly the right proportions to be biologically active. The female effluvia or sex gland can contain additional compounds which are related to the pheromone components and whose biological function is often unclear. On the other hand, many attractants of male moths have been discovered simply by field screening. In several cases it could later be shown that the attractant found with this technique was identical to the natural pheromone produced by the female. In most others, the composition of the true pheromone is still unknown.

While it is known that pheromone systems exist in insects, an increasing number of studies have shown that pheromones play a role in many species, including humans. Pheromones in humans are believed to be produced by the apocrine glands. These glands become functional after reaching puberty, which could explain why most people develop an attraction for others at that time. Pheromones could also be the reason why a person can sense “chemistry”, or feel an instant attraction or dislike when first meeting someone.

Certain compounds believed to have pheromone properties affect a specific behavioral or physiological response in human subjects, e.g., a reduction of negative affect, mood, and character traits. In one particular example, nasal administration provides for contacting neurochemical receptors of a heretofore poorly understood neuroendocrine structure, commonly known as the vomeronasal organ (“VNO”, also known as “Jacobson's Organ”), with one or more steroid(s) or with compositions containing the steroid(s). This organ is accessed through the nostrils of most higher animals—from snakes to humans, and has been associated, inter alia, with pheromone reception in certain species (see generally Muller-Schwarze & Silverstein, Chemical Signals, Plenum Press, New York (1980)). The axons of the neuroepithelia of the vomeronasal organ, located supra palatinal, form the vomeronasal nerve and have direct synaptic connection to the accessory olfactory bulb and indirect input from there to the cortico-medial amygdaloid basal forebrain and hypothalamic nuclei of the brain. The distal axons of terminalis nerve neurons may also serve as neurochemical receptors in the VNO. Stensaas, L. J., et al., J. Steroid Biochem. and Molec. Biol. (1991) 39:553. This nerve has direct synaptic connection with the hypothalamus.

### SUMMARY OF THE INVENTION

The invention provides confectionary products and food items, such as candies, which are formulated with and/or coated with a human pheromone substance. The confectionary products of the present invention will cause the person eating the confectionary product to experience a unique, mood-altering effect. This effect may also be an overall sense of well-being or may one of heightened sexual attractiveness.

### DESCRIPTION OF THE INVENTION

As used herein, a “pheromone” is a substance that provides chemical means of communication between members of the same species through secretion and peripheral chemoreception. In mammals pheromones are usually detected by receptors in the vomeronasal organ of the nose. Commonly, pheromones effect development, reproduction and related behaviors. The pheromone compositions according to the present invention are intended to cause a change in human hypothalamic function, thereby altering certain behavior and physiology mediated by the hypothalamus of individuals.

As used herein, “confectionary” is intended to refer to sweet preparations, candies and confections.

The confectionary product of the present invention comprises an edible candy matrix and at least one human pheromone component. As used herein, “matrix” is intended to refer to the candy base formed from the various ingredients.

In addition to physiological responses, pheromones can be identified by their species specific binding to receptors in the vomeronasal organ (VNO). Thus, human pheromones bind to human receptors. This can be demonstrated by measuring the change in the summated potential of neuroepithelial tissue in the presence of the pheromone. Human pheromones induce a change of at least about −5 millivolts in human neuroepithelial tissue of the appropriate sex (The binding of pheromones is generally sexually dimorphic, i.e., different in the effect of, or response to, a compound or composition between males and females of the same species). Naturally occurring human pheromones induce sexually dimorphic changes in receptor binding potential in vivo in the human VNO. Naturally occurring human pheromones can be extracted and purified from human skin and they can also be synthesized. “Human pheromones” are pheromones which are naturally occurring in humans and effective as a specifically binding ligand in human VNO tissue, regardless of how the pheromone was obtained. Thus, both a synthesized and purified molecule may be considered a human pheromone.

In one embodiment, chocolate candy is provided with a pheromone ingredient or which has a coating which includes a human pheromone compound. The human pheromone is generally colorless, tasteless and odorless and thus does not detract in any way from the appearance, smell or taste of the chocolates. Since human pheromones are effective in very low quantities, the amount added to the chocolate candy and other confectionary products of the present invention may be in picogram quantities.

In another embodiment the pheromone composition will be incorporated into an inner portion of a chocolate candy. In a solid chocolate, the human pheromone may be incorporated directly into the chocolate mixture which solidifies and is then shaped or sized for an appropriate serving size. The human pheromone may also be incorporated into an internal liquid filling such as, but not limited to, caramel, nougat, toffee, syrup or other flavored pastes or gels dispersed within the chocolate.

The invention is not in any way limited to a specific type of chocolate. All chocolates, such as milk chocolate, dark or bittersweet chocolate, fudge, and white chocolate are all contemplated by the present invention. Additionally, low-fat and low-caloric chocolates are contemplated and may be formulated with one or more human pheromones. The invention may also include added ingredients such as nuts, raisins, rice, cereal, dried fruit.

The chocolate product manufactured in accordance with the present invention may be provided in a variety of forms, shapes and sizes. Bars or bite-sized single serving pieces may be made in accordance with the process described below. The bite-sized pieces may be individually wrapped or may be part of an assortment of numerous chocolates of the same or of different types or flavors.

In another embodiment the pheromone composition will be incorporated into or coated upon hard candy confections. The hard candy confections may be on a stick, such as a lollipop or may be in the form of a drop which, when placed into the mouth, dissolves by sucking. Hard candy confections are amorphous confections having a relatively smooth and glassy surface. They appear in various shapes and sizes and come in various flavors, such as fruit, mint, butterscotch, peppermint, and the like. As used herein, the term “amorphous” refers to the “glassy” or “non-crystalline” state of the hard candy.

Soft candies such as chewable caramels, taffy, and gums are also contemplated by the present invention and are all easily prepared with one or more human pheromone compounds in accordance with the present invention.

The chocolates and hard and soft candies in accordance with the present invention exhibit mouthfeel, texture and organoleptic characteristics typical of other chocolate and hard and soft candies respectively.

The present invention is not limited in the pheromone composition used. Exemplary pheromone compounds include, but are not limited to, copulins (such as short chain aliphatic acids), estrene steroids, vomeropherins, such as 19-nor-cholane steroids, 19-nor-pregnane steroids such, and androstene and androstenedione compounds. Metabolites of the above-recited human pheromone compounds are also contemplated.

Upon the VNO sensing of the pheromone in the confectionary product of the present invention, the human pheromone will exert its effect by altering hypothalamic function of an individual. Such alteration of hypothalamic function will bring about physiological and behavioral functions of individuals which are normally regulated by the hypothalamus. The changes in physiological functions may cause the individual eating the confectionary product to experience a unique, mood-altering effect or an overall sense of well-being and/or heightened sexual attractiveness.

It has been found that certain human pheromones have a distinct effect on men and others on women, i.e., the compounds are sexually dimorphic. As such, the present invention is not limited to the use of a specific pheromone compound within the confection product and it is contemplated that certain confectionary products directed for men will contain a human pheromone compound different from the confectionary product directed for women or that a single product may contain a mix of human pheromone compounds.

### EXAMPLES

### Example 1

Process of Making Pheromone-Containing Chocolate

First, cocoa beans are milled and ground to form a chocolate liquor, a liquefied form of pure chocolate flavor. The main ingredients in chocolate are the chocolate liquor, cocoa butter, sugar and milk. Chocolate liquor is a liquefied form of milled and grinded cocoa beans. These ingredients are mixed together and then dried into a coarse powder known as chocolate crumb. One or more pheromones may be added to this mixture in forming the chocolate crumb. Cocoa butter is then added to the powder to bring out the rich taste and creamy texture of the chocolate. The crumb then travels through special steel rollers which grind and refine the mixture, making it smoother. The crumb becomes a thick liquid called chocolate paste. The paste is poured into huge vats called conches. Once inside the conche, large granite rollers smooth out the gritty particles from the crumb. This process can take anywhere from 24 to 72 hours to complete. At this point, the chocolate paste has the smooth, familiar look of milk chocolate and it's ready to be molded into bars, bite-sized morsels, etc. The paste is then tempered, or cooled in a controlled manner to the right texture and consistency. Other ingredients, like almonds or peanuts, can be mixed into the paste during tempering or added directly to the molds.

Process of Making Pheromone-Containing Lollipops

If molds are being used, the molds are prepared with lollipop sticks and sprayed with oil. Over heat sugar, corn syrup, water, cream of tarter (potassium bitartrate) and other ingredients may be combined until the sugar crystals dissolve. This is commonly stirred to dissolve the sugar crystals clinging to the sides of the container and then as the syrup begins to boil the stirring is stopped. The syrup is brought to the hard-crack stage which is commonly around 300 degrees. The syrup is then removed from the heat and cooled to about 275 degrees before adding flavor, color, and citric acid. One or more pheromone compositions are also added at this time. The syrup is then poured into prepared molds and cooled.

While there has been shown and described what is considered to be preferred embodiments of the invention and two examples, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A confectionary product, comprising:
(a) an edible candy matrix; and
(b) a human pheromone compound.

2. The confectionary product as recited in claim 1, wherein said candy matrix is a chocolate matrix.

3. The confectionary product as recited in claim 1, wherein the candy matrix is an amorphous hard candy.

4. The confectionary product as recited in claim 1, wherein the candy matrix is a soft candy.

5. The confectionary product as recited in claim 1, wherein said human pheromone compound is selected from the group consisting of 19-nor-cholane compounds, 19-nor-pregnane compounds, estrene steroids, short chain aliphatic acids, androstadienone steroids, androstene, androstane and androstenedione compounds, combinations thererof and metabolites thereof.

6. The confectionary product as recited in claim 1, comprising two or more human pheromone compounds.

7. A chocolate confectionary product comprising one or more human hormone compounds selected from the group consisting of 19-nor-cholane compounds, 19-nor-pregnane compounds, estrene steroids, short chain aliphatic acids, androstadienone steroids, androstene, androstane and androstenedione compounds, combinations thererof and metabolites thereof.

8. An amorphous flavored hard candy confection comprising one or more human hormone compounds selected from the group consisting of 19-nor-cholane compounds, 19-nor-pregnane compounds, estrene steroids, short chain aliphatic acids, androstadienone steroids, androstene, androstane and androstenedione compounds, combinations thererof and metabolites thereof.

9. The hard candy as recited in claim 8, wherein said hard candy is secured on a stick.

10. The hard candy as recited in claim 8, wherein said candy is sized for individual wrapping and consumption.

11. A process for preparing a candy product with a human pheromone component, the process comprising the steps of:
(a). preparing a chocolate liquor milled cocoa beans;
(b). adding cocoa butter, sugar, milk and one or more pheromone compounds to the chocolate liquor;
(c). mixing the ingredients in step (b);
(d). drying the mixture of step (c) to form a powder;
(e). adding cocoa butter to the powder formed in step d;
(f). grinding the mixture to a desired smoothness to form a paste;
(g). pouring the paste into conches where rollers smooth the gritty particles in the paste;
(h). molding the paste into a desired size; and
(i). cooling to a desired texture and consistency.

12. The process as recited in claim 11, wherein said one or more human pheromone is selected from the group consisting of 19-nor-cholane compounds, 19-nor-pregnane compounds, estrene steroids, short chain aliphatic acids, androstadienone steroids, androstene, androstane and androstenedione compounds, combinations thererof and metabolites thereof.

13. The process as recited in claim 11, wherein the grinding step (f) further comprises the step of adding additional flavoring ingredients.

14. The process as recited in claim 11, wherein said additional flavoring ingredients are selected from the group consisting of nuts, raisins, coconut, nougat, caramel, or jelly
